(19) United States
(12) Patent Application Publication
(10) Pub. No.: US 2026/0207316 A1
(43) Pub. Date: Jul. 23, 2026

(54) PACKAGING ASSEMBLY FOR DELIVERY APPARATUS FOR IMPLANTABLE MEDICAL DEVICE

(71) Applicant: Edwards Lifesciences Corporation, Irvine, CA (US)

(72) Inventors: Vipul P. Rajpara, Lake Forest, CA (US); Sameer Dinmanishanker Upadhyaya, Irvine, CA (US)

(21) Appl. No.: 19/569,942

(22) Filed: Mar. 17, 2026

Related U.S. Application Data
(63) Continuation of application No. PCT/US2024/047441, filed on Sep. 19, 2024.
(60) Provisional application No. 63/584,301, filed on Sep. 21, 2023.

(51) Int. Cl. A61F 2/00; A61F 2/24

(52) U.S. Cl. CPC A61F 2/0095; A61F 2/2427

(57) ABSTRACT

An assembly can comprise a delivery apparatus, a prosthetic valve, and a packaging. The delivery apparatus can comprise a proximal end portion and a distal end portion, wherein the proximal end portion comprises a first connector. The prosthetic heart valve can be crimped around the distal end portion of the delivery apparatus. The packaging can include a hollow body comprising a proximal end portion and a distal end portion. The delivery apparatus and prosthetic heart valve crimped thereunto can be disposed within the hollow body. The packaging can further include a retainer comprising a second connector configured to detachably engage the first connector of the delivery apparatus.

### CROSS-REFERENCE TO RELATED APPLICATION

This application is a continuation of PCT Patent Application No. PCT/US2024/047441 filed on Sep. 19, 2024, which claims the benefit of U.S. Provisional Patent Application Ser. No. 63/584,301 , filed Sep. 21, 2023, each of these applications being incorporated by reference herein in its entirety.

### FIELD

The present disclosure relates to packaging for delivery apparatuses for implantable medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

In some examples, the delivery apparatus is packaged before use. Accordingly, a need exists for improved delivery apparatus packaging.

### SUMMARY

Described herein is packaging for containing a delivery apparatus for an implantable medical device, such as a prosthetic heart valve.

In some examples, an assembly can comprise a delivery apparatus, a packaging assembly, and optionally a prosthetic heart valve.

In some examples, the delivery apparatus can comprise a handle and one or more shafts coupled to the handle.

In some examples, the delivery apparatus can comprise a catheter balloon mounted on a distal end portion of one of the shafts.

In some examples, the delivery apparatus can comprise a proximal end portion comprising a first connector.

In some examples, the prosthetic heart valve can be configured to be crimped around the catheter balloon of the delivery apparatus.

In some examples, the packaging assembly can comprise a hollow body comprising a proximal end portion and a distal end portion.

In some examples, the packaging assembly can comprise a retainer configured to detachably engage both the hollow body and the delivery apparatus.

In some examples, the retainer can comprise a second connector configured to detachably engage the first connector of the delivery apparatus.

In some examples, the delivery apparatus and prosthetic heart valve crimped thereunto can be disposed within the hollow body.

In some examples, the hollow body can comprise at least one first locking feature.

In some examples, the retainer can comprise at least one complementary second locking feature configured to engage the first locking feature.

In some examples, the at hollow body can comprise a plurality of first locking features.

In some examples, the plurality of first locking features can be spaced apart from each other in a circumferential direction.

In some examples, the first connector can be a port of a threaded Y-connector.

In some examples, the port can comprise a lumen for receiving a guidewire.

In some examples, the port can comprise external threads for engaging the second connector.

In some examples, the distal end portion of the delivery apparatus can be suspended within the hollow body when the retainer is engaged with both the delivery apparatus and the hollow body and when the packaging and the delivery apparatus are placed in a vertical orientation with the proximal end portion of the hollow body above the distal end portion of the hollow body.

In some examples, the assembly can comprise a balloon cover.

In some examples, an assembly can comprise a delivery apparatus assembly, a prosthetic heart valve, and a packaging. The delivery apparatus can comprise a proximal end portion and a distal end portion, wherein the proximal end portion comprises a first connector. The prosthetic heart valve can be crimped around the distal end portion of the delivery apparatus. The packaging can comprise a hollow body comprising a proximal end portion and a distal end portion and a retainer comprising a second connector configured to detachably engage the first connector of the delivery apparatus. The delivery apparatus and prosthetic heart valve crimped thereunto can be disposed within the hollow body.

In some examples, a packaging assembly for a delivery apparatus for an implantable medical device can comprise a tube structure and a first sealing member. The tube structure can comprise a proximal end portion defining a proximal end, a distal end portion defining a distal end, an inner surface, an outer surface, and at least one aperture. The first sealing member can cover the aperture, wherein the first sealing member can be vapor-permeable.

In some examples, an assembly can comprise a delivery apparatus, an implantable medical device coupled to the delivery apparatus, and a packaging assembly for packing the delivery apparatus and implantable medical device therein. The packaging assembly can comprise a tube structure and a retainer detachably coupled to an end portion of the tube structure. The retainer can be configured to engage a proximal end portion of the delivery apparatus. In some examples, a distal end portion of the delivery apparatus does not directly contact any other portion of the packaging assembly when the retainer engages the proximal end portion of the delivery apparatus and when the delivery apparatus is disposed entirely within the tube structure.

In some examples, a packaging assembly for a delivery apparatus for an implantable medical device can comprise a tube structure and a retainer. The tube structure can comprise a proximal end portion defining a proximal end, a distal end portion defining a distal end, an inner surface, an outer surface, and at least one first locking feature disposed on the inner surface along the proximal end portion. The retainer can comprise at least one second locking feature and a connector. The second locking feature can be configured to engage the first locking feature. The connector can be configured to be releasably connected to a proximal end portion of the delivery apparatus.

In some examples, a delivery apparatus comprises one or more of the components recited in Examples 1-45 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of a delivery apparatus for a prosthetic heart valve, according to one example.

FIG. 3 is a partially exploded, perspective view of a medical assembly comprising a prosthetic heart valve, a delivery apparatus, and a packaging assembly, according to one example.

FIG. 4 is a top-down view of a tube structure of the packaging assembly of FIG. 3.

FIG. 5A is a perspective view of a retainer of the packaging assembly of FIG. 3.

FIG. 5B is a bottom plan view of the retainer of FIG. 5A.

FIG. 6A is a perspective view of a cap of the packaging assembly of FIG. 3.

FIG. 6B is another perspective view of the cap of FIG. 6A.

FIG. 7 is a side view of a balloon cover of a packaging assembly, according to one example.

FIG. 8A is a side view of a delivery apparatus being inserted into a packaging assembly, according to one example.

FIG. 8B is a side view of the delivery apparatus and the packaging assembly of FIG. 8A after the delivery apparatus is inserted into the packing assembly and a retainer of the packaging assembly engages a tube structure of the packaging assembly, according to one example.

FIG. 8C is a side view of the packaging assembly and delivery apparatus of FIGS. 8A and 8B being sterilized, according to one example.

FIG. 8D is a side view of the packaging assembly of FIGS. 8A-8C after being sealed, according to one example.

FIG. 8E is a side view of the packaging assembly of FIGS. 8A-8C partially filled with a hydrating solution, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Described herein are examples of assemblies that include delivery apparatuses that can be used in various medical procedures and packaging therefor. In some examples, the disclosed assemblies can comprise a delivery apparatus that can be used to navigate a subject's vasculature to deliver an implantable, expandable medical device (for example, a prosthetic heart valve), tools, agents, or other therapy to a location within the body of a subject. Examples of procedures in which the delivery apparatuses are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

A delivery apparatus is typically packaged within a packaging assembly for shipment and storage before use at a medical clinic. A prosthetic heart valve to be used with the delivery apparatus typically is stored in a separate package or container, such as storage jar, which also contains a liquid, such as glutaraldehyde solution, to help preserve any tissue-based components of the prosthetic heart valve. Prior to a procedure, the physician removes the delivery apparatus and the prosthetic valve from their respective packaging and loads the prosthetic valve onto the delivery apparatus (for example, by crimping the prosthetic valve onto an inflatable balloon of the delivery apparatus). Thus, most known packaging assemblies are not equipped for use with a delivery apparatus and a prosthetic heart valve pre-loaded on the delivery apparatus.

Accordingly, in some examples of the present disclosure, a delivery apparatus packaging assembly can be configured to store both a delivery apparatus and an implantable medical device (for example, a prosthetic heart valve) therein.

In some examples, a delivery apparatus and/or implantable medical device is sterilized within a packaging assembly before the packaging assembly is sealed.

In some examples, a packaging assembly helps ensure that the delivery apparatus and/or implantable medical device stored therein are stored under proper environmental conditions.

In some examples, a packaging assembly facilitates rehydration of the implantable medical device stored therein prior to use in an implantation procedure.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

### Example Prosthetic Heart Valve

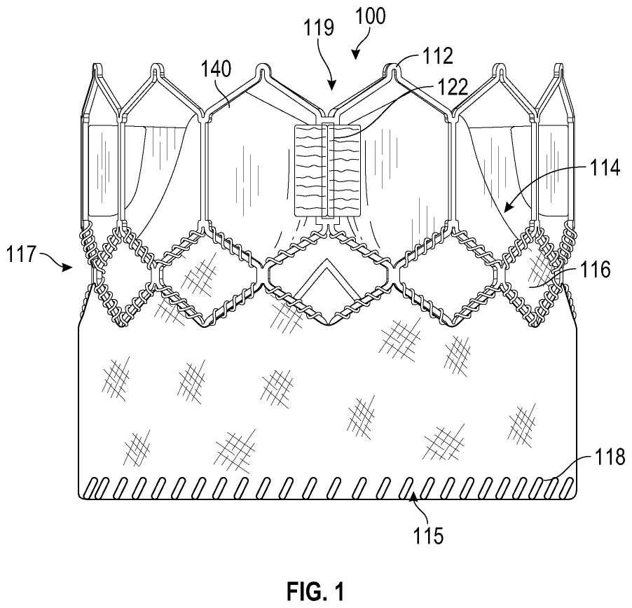

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 100 can comprise a frame 112, a valvular structure 114, an inner skirt 116, and a perivalvular outer sealing member or outer skirt 118. The prosthetic valve 100 can comprise an inflow end portion 115 and an outflow end portion 119, and an intermediate portion 117 extending therebetween.

The valvular structure 114 can comprise a plurality of leaflets 140 collectively forming a leaflet structure. In some examples, the valvular structure 114 can comprise three leaflets 140 arranged in a tricuspid arrangement. However, there can be a greater or fewer number of leaflets 140. The leaflets can be secured to one another at their adjacent sides to form commissures 122 of the valvular structure 114. The lower edge of the valvular structure 114 can have an undulating, curved scalloped shape, and can be secured to the inner skirt 116 by sutures (not shown). In some examples, the leaflets 140 can be formed of pericardial tissue (such as bovine pericardial tissue), biocompatible synthetic materials, or other various suitable natural or synthetic materials as known in the art and described in U.S. Pat. No. 6,730,118, which is incorporated by reference herein.

In some examples, the leaflets 140 (typically pericardial tissue) can be treated during the manufacturing process so that they are completely or substantially dehydrated and can be stored in a partially or fully crimped state with a hydrating fluid. Methods for treating tissue leaflets for dry storage are disclosed in U.S. Pat. No. 8,007,992 and U.S. Patent Application Publication No. 2009/0164005, filed Dec. 18, 2008, both of which are incorporated by reference herein in their entireties. In some examples, the packaging assemblies described herein can be used to store a delivery apparatus and a prosthetic heart valve in a dry environment, as further described below. One specific example of the prosthetic heart valve 100 that can be stored in a dry environment using the packaging assemblies disclosed herein is the Sapien 3 Ultra Resilia valve sold by Edwards Lifesciences Corporation (Irvine, California).

The frame 112 can be made of any of various suitable plastically-expandable materials (for example, stainless steel, etc.) or self-expanding materials (for example, Nitinol) as known in the art. When constructed of a plastically-expandable material, the frame 112 (and thus the valve 100) can be crimped to a radially compressed state on a delivery catheter and then expanded inside a patient by an inflatable catheter balloon or equivalent expansion mechanism. When constructed of a self-expandable material, the frame 112 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 112) include, metal alloys, polymers, or combinations thereof. Example metal alloys can comprise one or more of the following: nickel, cobalt, chromium, molybdenum, titanium, or other biocompatible metal. In some examples, the frame 112 can comprise stainless steel. In some examples, the frame 112 can comprise cobalt-chromium. In some examples, the frame 112 can comprise nickel-cobalt-chromium. In some examples, the frame 112 comprises a nickel-cobalt-chromium-molybdenum alloy, such as MP35N™ (tradename of SPS Technologies), which is equivalent to UNS R30035 (covered by ASTM F 562-02). MP35N™/UNS R30035 comprises 35% nickel, 35% cobalt, 20% chromium, and 10% molybdenum, by weight.

The inner skirt 116 and/or the outer skirt 118 can be wholly or partly formed of any suitable biological material, synthetic material (for example, any of various polymers), or combinations thereof. In some examples, the skirts 116, 118 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirts 116, 118 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirts 116, 118 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirts 116, 118 can comprise a sponge material or foam, such as polyurethane foam. In some examples, the skirts 116, 118 can comprise natural tissue, such as pericardium (for example, bovine pericardium, porcine pericardium, equine pericardium, or pericardium from other sources).

### Example Delivery Apparatus

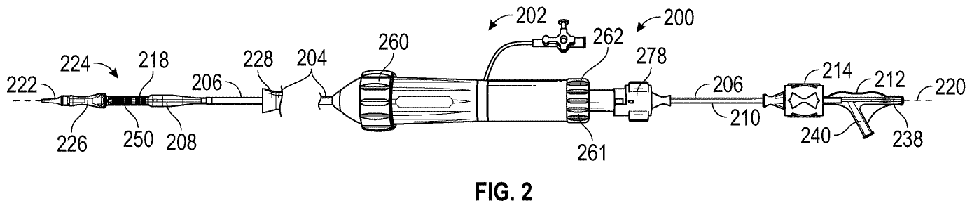

FIG. 2 shows a delivery apparatus 200, according to one example, in the form of a balloon catheter that can be used to implant a prosthetic medical device. In some examples, the delivery apparatus 200 can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic heart valve into a heart.

The delivery apparatus 200 in the illustrated example of FIG. 2 comprises a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic heart valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable catheter balloon 218 (which also referred to herein as a “balloon”) of the delivery apparatus 200. A distal end portion of the inner shaft 208 can extend distally beyond the distal end portion of the intermediate shaft 206.

The catheter balloon 218 can be coupled to the distal end portion of the intermediate shaft 206.

In some examples, a distal end of the catheter balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the catheter balloon 218 can overlay a valve mounting portion 224 of a distal end portion of the delivery apparatus 200 and a distal end portion of the catheter balloon 218 can overly a distal shoulder 226 of the delivery apparatus 200. The valve mounting portion 224 and the intermediate portion of the catheter balloon 218 can be configured to receive a prosthetic heart valve in a radially compressed state. For example, as shown schematically in FIG. 2, a prosthetic heart valve 250 (which can be one of the prosthetic heart valves described herein) can be mounted around the catheter balloon 218, at the valve mounting portion 224 of the delivery apparatus 200.

The balloon shoulder assembly, including the distal shoulder 226, is configured to maintain the prosthetic heart valve 250 (or other prosthetic medical device) at a fixed position on the catheter balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic heart valve 250 is mounted in the radially compressed state on the valve mounting portion 224 (as shown in FIG. 2) and during delivery of the prosthetic heart valve to the target implantation site. As such, the distal tip portion 228 can be configured to resist movement of the prosthetic heart valve 250 relative to the catheter balloon 218 proximally, in the axial direction, relative to the catheter balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the catheter balloon 218. As such, fluid from the fluid source can flow to the fluid passageway from the annular space to inflate the catheter balloon 218 and radially expand and deploy the prosthetic heart valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Publication No. WO2022/046585, which is incorporated by reference herein.

### Example Packaging Assembly

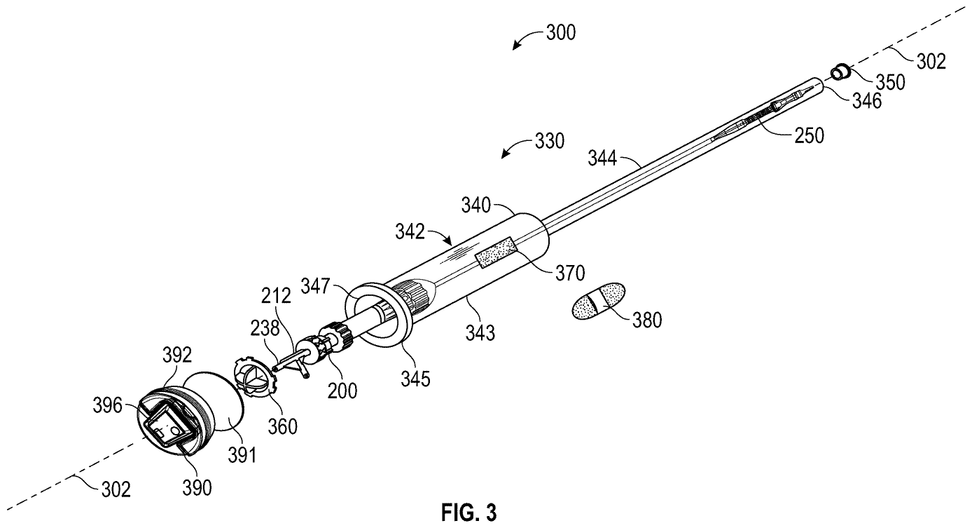

FIG. 3 is a partially exploded, perspective view of a medical assembly 300, according to one example. As shown, the medical assembly 300 (which is also referred to herein as an “assembly”) comprises the prosthetic heart valve 250 (which, in some examples, can be the same or similar to the prosthetic heart valve 100 of FIG. 1, another type of prosthetic heart valve, or another type of implantable medical device), the delivery apparatus 200 (which, in some examples, can be any of the other delivery apparatus described herein, or another delivery apparatus), and a packaging assembly 330 configured to store the prosthetic heart valve 250 and the delivery apparatus 200 therein. Thus, it should be understood that the packaging assembly 330 is a sub-assembly of the overall medical assembly 300. The following description proceeds with reference to using a prosthetic heart valve with the medical assembly 300. However, it should be understood that the prosthetic heart valve 250 in the medical assembly 300 can be replaced with any implantable, expandable, and/or prosthetic medical device (for example, a stent, a graft, an embolic coil, to name a few).

As shown, the delivery apparatus 200 comprises the proximal end portion and the distal end portion, wherein the proximal end portion of the delivery apparatus 200 comprises the adaptor 212 with the first port 238. The adaptor 212 is illustrated as a Y-connector. The first port 238 can serve as a first connector of the delivery apparatus 200 that mates with a corresponding second connector of the packaging assembly 330 and is thus also referred to herein as a “first connector.” For example, the first port 238 can comprise external threads configured to engage corresponding internal threads on the second connector 368 of the packaging assembly 330, such that the first port 238 can be coupled to the second connector 368.

As shown, the packaging assembly 330 (which is also referred to herein as a “packaging” and/or a “package”) comprises a tube structure 340, a plug 350 coupled to a distal end portion of the tube structure 340, a retainer 360 detachably coupled to a proximal end portion of the tube structure 340, and an optional first sealing member 370 configured to cover an aperture of the tube structure 340. In some examples, the packaging assembly 330 can optionally comprise a second sealing member 380 configured to cover the aperture of the tube structure 340 and the first sealing member 370. The packaging assembly 330 can further comprise a cap 390 detachably coupled to a proximal end portion of the tube structure 340.

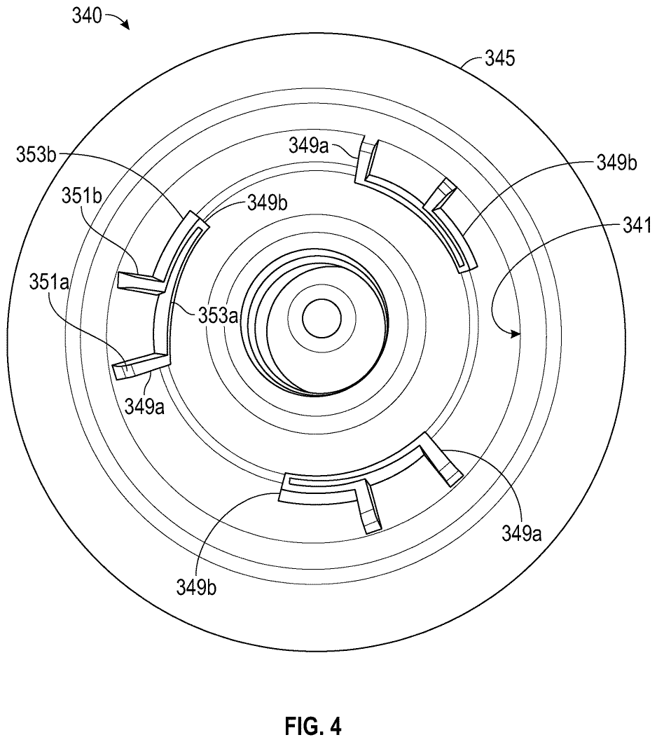

The tube structure 340 can comprise a hollow body defining an inner surface 341 (FIG. 4), an outer surface 342, and a cavity bounded by the inner surface 341. The cavity of the tube structure 340 can be sized and/or configured to accommodate the delivery apparatus 200 and the prosthetic heart valve 250 coupled to the distal end portion of the delivery apparatus 200 therein. For example, the cavity of the tube structure 340 can be sized to accommodate the prosthetic heart valve 250 and the delivery apparatus 200 coupled together in a “pre-crimped configuration.” Storing the prosthetic heart valve 250 in the pre-crimped configuration on the delivery apparatus 200 can beneficially simplify and streamline the implantation procedure by eliminating the need for a separate “crimping” step performed by the physician. Instead, the prosthetic heart valve 250 can be pre-crimped on the delivery apparatus 200 at a manufacturing facility, after which the delivery apparatus 200 and the prosthetic heart valve 250 are inserted into the packaging assembly 330 and then shipped to a medical facility for storage until use in the implantation procedure.

The tube structure 340 can comprise one or more tube segments aligned along a central longitudinal axis 302 of the medical assembly 300 and coupled together to form the hollow body. For example, the illustrated tube structure 340 comprises a proximal tube segment 343 forming a proximal portion of the tube segment 340 and a distal tube segment 344 forming a distal portion of the tube segment 340. A proximal-most end portion of the proximal tube segment 343 can define a proximal end portion of the tube structure 340 and a distal-most end portion of the distal tube segment 344 can define a distal end portion of the tube structure 340. The proximal end portion of the tube structure 340 can define a proximal end 345 of the tube structure 340 and the distal end portion of the tube structure 340 can define a distal end portion 346 of the tube structure 340. In some examples, the tube segments 343 and 344 can be separately formed and subsequently attached to each other, in which the tube segments 343 and 344 can be hermetically sealed to each other, such as by welding, to prevent the ingress of contaminants into the tube structure 340 and/or prevent fluids from leaking out of the tube structure 340. In other examples, the entire tube structure 340 can be formed as a unitary component including the tube segments 343 and 344, such as by injection molding or other known techniques.

In some examples, the proximal tube segment 343 can have a diameter (perpendicular to the central longitudinal axis 302) greater than the diameter of the distal tube segment 344. Since the proximal portion of the delivery apparatus 200, which includes the handle 202, can be wider in diameter than the distal portion of the delivery apparatus 200 which includes a catheter balloon 218 and nose cone 222, sizing the proximal tube segment 343 and the distal tube segment 344 relative to the portions of the delivery apparatus 200 received in those segments can beneficially minimize the amount of empty space within the tube structure 340 when the prosthetic heart valve 250 and the delivery apparatus 200 are inserted therein.

In some examples, the diameter of the distal tube segment 344 can be wider than the diameter of the prosthetic heart valve 250 in a crimped or compressed configuration and/or the diameter of the distal end portion of the delivery apparatus 200. For example, the distal end portion of the delivery apparatus 200 and/or the prosthetic heart valve 250 crimped thereunto can be spaced apart in a radial direction (perpendicular to the central longitudinal axis 302) from the inner surface 341 of the tube structure 340 (and more specifically, the inner surface of the distal tube segment 344). Furthermore, in some examples, the distal end portion of the delivery apparatus 200 and/or the prosthetic heart valve 250 crimped thereunto can be spaced apart in a longitudinal direction (parallel to the central longitudinal axis 302) from the distal end 346 of the tube structure 340 and/or the plug 350 coupled to the distal end 346 of the tube structure 340. In such examples, it can be beneficial to provide clearance between the tube structure 340 and the prosthetic heart valve 250 and/or delivery apparatus 200 stored therein to avoid or minimize contact between the prosthetic heart valve and the inner surface 341.

In some examples, the tube structure 340 and/or any portion thereof can be formed from a rigid material, such as a rigid polymer. For example, each of the proximal tube segment 343 and the distal tube segment 344 can be formed from a rigid material to prevent the tube structure 340 from bending and damaging the prosthetic heart valve 250 and the delivery apparatus 200 stored therein.

In the illustrated example, the tube structure 340 (including proximal and distal tube segments) are cylindrical. However, other shapes are possible. For example, a cross-sectional profile of the tube structure 340 (the cross-sectional profile taken perpendicular to the longitudinal axis 302) can be any of various shapes, including square, rectangular, triangular, oval, circular, and/or any combination thereof. Moreover, the tube structure 340 in the illustrated example is straight and defines an interior cavity sized to contain the delivery apparatus 200 in a straight configuration without any bends.

In some examples, the tube structure 340 can comprise a proximal aperture or opening 347 disposed at the proximal end 345. The normal direction of the proximal aperture 347 can be parallel to the central longitudinal axis 302. In some examples, the proximal aperture 347 can be sized to allow the ingress and/or egress of the prosthetic heart valve 250 and the delivery apparatus 200 therethrough. For example, the diameter of the proximal aperture 347 can be the same as the inner diameter of the proximal tube segment 343.

In some examples, the tube structure 340 can optionally comprise at least one intermediate aperture (which is also referred to herein as an “intermediate vent” and/or an “intermediate sealable vent”) disposed between the proximal end 345 and the distal end 346 of the tube structure 340 and extending from the inner surface 341 to the outer surface 342 (for example, in a direction perpendicular to the central longitudinal axis 302). The intermediate aperture can be configured to allow for the ingress of sterilization fluids to facilitate the sterilization of the prosthetic heart valve 250 and/or the delivery apparatus 200 stored within the tube structure 340.

In some examples, the tube structure 340 can comprise a first sealing member 370 (which is also referred to herein as a “fixed sealing member and/or “first vent cover”), wherein the first sealing member 370 can be configured to cover the intermediate aperture. In some examples, the first sealing member 370 can be configured to be fixed (or not detachably coupled to) the tube structure 340. In some examples, the first sealing member 370 can be vapor-permeable to allow for the ingress of sterilization fluids. In some examples, the first sealing member 370 can be formed from any vapor-permeable material (represented by stippling), such as Tyvek.

In some examples, the tube structure 340 can optionally comprise a second sealing member 380 (which is also referred to herein as a “removable sealing member,” a “second vent cover,” an “adhesive vent cover,” and/or a “removable vent cover”). The second sealing member 380 can be configured to hermetically seal the intermediate aperture by covering the intermediate aperture and/or the first sealing member 370. The second sealing member 380 can be sized to extend over and completely cover the first sealing member 370 as well an adjacent portion of the outer surface 342 of the tube structure surrounding the first sealing member 370. The second sealing member 380 can comprise an adhesive cover that can be adhered to the adjacent surface portion of the tube structure and optionally the first sealing member 370, thereby forming a hermetic seal with the adjacent portions of the tube structure 340. In some examples the second sealing member 380 can be adhered to the tube structure 340 to prevent contaminants from entering the tube structure 340 after the contents of the packaging assembly 330 have been sterilized. In some examples, the second sealing member 380 can be peeled off the tube structure 340 to expose the intermediate aperture and/or the first sealing member 370, for example, to allow for the ingress of sterilization fluids and/or fluids for rehydrating the prosthetic heart valve 250.

The plug 350 can be coupled to the distal end 346 of the tube structure 340. For example, the plug 350 can be coupled to the distal-most end portion of the distal tube segment 344. In some examples, the distal end portion of the delivery apparatus 200 and/or the prosthetic heart valve 250 crimped thereunto can be spaced apart in the longitudinal direction (parallel to the central longitudinal axis 302) from the plug 350 to prevent the plug 350 from contacting and/or damaging the prosthetic heart valve 250 and/or the distal end portion of the delivery apparatus 200. In some examples, the plug 350 can be configured to form a fluid-tight seal and/or a hermetic seal with an opening at the distal end 346 of the tube structure 340.

Now referring to FIG. 4, the tube structure 340 can comprise at least one first locking feature 349 (which is also referred to herein as a “locking slot,” a “locking recess,” and/or a “locking channel”) disposed on the inner surface 341 of the tube structure 340. As shown, the tube structure 340 comprises three locking features 349 spaced apart in a circumferential direction of the delivery apparatus assembly 330, but some examples of the tube structure 340 can comprise any number of first locking features 349 (for example, one, two, four, five, six, seven, eight, etc.). The first locking feature 349 can be disposed at the proximal end portion of the tube structure 340. In some examples, the first locking feature 349 can comprise a locking slot, at least a portion of which can extend in the longitudinal direction of the medical assembly 300. As shown, the first locking feature 349 can comprise a longitudinal portion 349a extending in the longitudinal direction and a circumferential portion 349b extending in the circumferential direction of the medical assembly 300. In some examples, the longitudinal portion 349a can be spaced apart in the longitudinal direction from the proximal end 345 of the tube structure 340 and, furthermore, some examples of the circumferential portion 349b can be spaced apart in the longitudinal direction from the proximal end 345 of the tube structure 340.

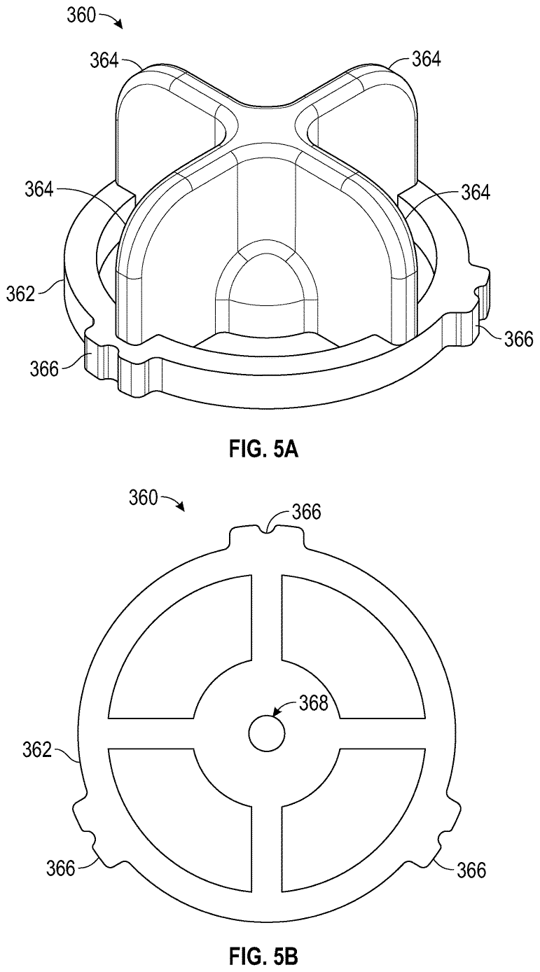

Each first locking feature 349 can comprise first and second longitudinal ribs 351a, 351b, respectively, forming the longitudinal portion 349a, and lower and upper circumferential ribs 353a, 353b, respectively, forming the circumferential portion 349b. The longitudinal ribs 351a, 351b define a longitudinally extending slot or channel that is in communication with a circumferentially extending slot or channel defined by the circumferential ribs 353a, 353b. Each first locking feature 349 is configured to receive a complementary second locking feature 366 (FIG. 5A-5B) of the retainer 360 to retain the retainer 360 in a locked position. For example, each second locking feature 366 can be initially inserted into the slot of the longitudinal portion 349a of the corresponding first locking feature 349 until the second locking feature 366 rests on the circumferential lower rib 353a. The retainer 360 is then rotated relative to the tube structure 340 to move the retainer 360 into a locked position, in which the second locking feature 366 can engage the circumferential portion 349b of the first locking feature 349, as further described below.

Now referring to FIG. 5A-5B, the retainer 360 can be configured to detachably engage the proximal end portion of the delivery apparatus 200 and the proximal end portion of the tube structure 340 to retain the delivery apparatus 200 within the tube structure 340. The retainer 360 can be rotated from an unlocked position (in which the retainer 360 can disengage the tube structure 340) and a locked position (in which the retainer 360 can engage the tube structure 340).

The retainer 360 can include an annular structure 362, at least one finger tab 364 extending in a proximal direction from the annular structure 362, at least one second locking feature 366 extending in a radially outwards-facing direction from the annular structure 362, and a second connector 368 coupled to the annular structure 362.

As shown, the retainer 360 comprises four finger tabs 364 extending in the proximal direction from the annular structure 362. A user can engage the finger tabs 364 to twist or rotate the retainer 360 in the circumferential direction relative to the rest of the packaging assembly 330 (for example, relative to the tube structure 340) to move the retainer 360 between the locked position and the unlocked position.

As shown, the retainer 360 comprises three second locking features 366 (which are also referred to herein as “locking lugs” and/or “locking tabs”) extending in the radially outwards-facing direction from the annular structure 362. The number of second locking features 366 can be the same as the number of the number of first locking features 349 on the tube structure 340. The second locking features 366 can be spaced apart in the circumferential direction of the medical assembly 300. Each locking feature 366 can comprise a radially outwards-facing lug or a tab configured to detachably engage the corresponding one of the first locking features 349.

The second connector 368 can be configured to detachably engage the proximal end portion of the delivery apparatus 200 (specifically, the first port 238 of the delivery apparatus 200). The second connector 368 can comprise a recess or aperture in a distal face of the retainer 360. The second connector 368 can further comprise internal threads within the recess or aperture, wherein the internal threads are configured to engage the corresponding external threads of the first port 238 of the delivery apparatus 200.

In some examples, the delivery apparatus 200 (and the prosthetic heart valve 250 coupled thereto) can be cantilevered or suspended within the tube structure 340 from the retainer 360 when these components are coupled together. For example, the distal end portion of the delivery apparatus 200 can be suspended within the tube structure 340 when the retainer 360 is engaged with both the delivery apparatus 200 and the tube structure 340 and when the medical assembly 300 is placed in a vertical orientation for storage. In the vertical orientation, the proximal end portion of the tube structure 340 can be above the distal end portion of the tube structure 340. In such an example, the only portion of the delivery apparatus 200 that engages the packaging assembly 330 can be the proximal end portion of the delivery apparatus 200 (for example, the first port 238). Such a suspended or cantilevered configuration can beneficially prevent the tube structure 340 from contacting the prosthetic heart valve 250 disposed therein.

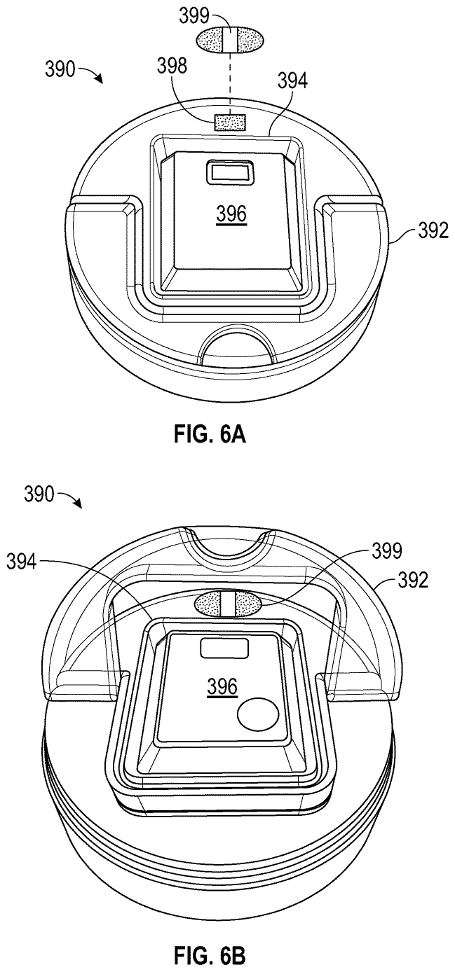

Now referring to FIGS. 6A-6B, the cap 390 comprises a structure configured to detachably engage and cover the proximal aperture 347 of the tube structure 340. The cap 390 can be further configured to cover the retainer 360 when the retainer 360 engages the tube structure 340. The cap 390 can comprise a hinged handle 392, a recess 394 in a proximal surface of the cap 390, and a sensor 396 disposed in the recess 394. The cap 390 can optionally comprise an aperture in the proximal surface of the cap 390, a first sealing member 398 configured to cover the aperture, and a second sealing member 399 configured to cover the aperture of the tube structure 340 and the first sealing member 398.

The cap 390 can be configured to detachably engage the proximal end portion of the tube structure 340. For example, the cap 390 can comprise internal threads configured to engage corresponding external threads on the exterior of the tube structure's proximal end portion 345 so that the cap 390 can be screwed onto and removed from the proximal end portion 345 of the tube structure 340. The cap 390 can form a hermetic seal with the tube structure 340 to prevent the ingress of contaminants and/or the egress of sterilization fluid and/or rehydration fluid.

The hinged handle 392 can comprise a semicircular hinge. In some examples, the hinged handle 392 can be used to hang the packaging assembly 330 (for example, from a rack in a medical clinic). The hinged handle 392 can beneficially facilitate the storage of the packaging assembly 330 in the suspended or cantilevered configuration. The hinged handle 392 can further beneficially facilitate the storage of the packaging assembly 330 in a vertical configuration that requires less floor space in an clinical environment.

The recess 394 can be configured to receive the sensor 396. In some examples, the sensor 396 can be any one or any combination of a temperature sensor, a humidity sensor, or any other sensor. For example, the sensor 396 can comprise a temperature sensor that monitors the temperature of the environment surrounding the packaging assembly 330 (and the prosthetic heart valve 250 disposed therein) during shipping and storage. In one specific example, the temperature sensor can be a TagAlert® Indicator sold by Sensitech, Inc. (Beverly, MA). In any of the aforementioned examples, the sensor 396 can help a user verify that the packaged prosthetic heart valve 250 has not been exposed to environmental conditions outside the range of acceptable storage conditions.

The aperture (which is also referred to herein as a “cap aperture,” a “vent,” a “sealable vent,” a “sealable cap vent,” and/or a “cap vent”) can comprise an aperture extending through the cap 390 from a proximal surface to a distal surface of the cap 390. The aperture can be configured to allow for the ingress of sterilization fluids into the tube structure 340 to facilitate the sterilization of the prosthetic heart valve 250 and/or the delivery apparatus 200 stored within the tube structure 340. The first sealing member 398 (which is also referred to herein as a “fixed sealing member and/or “first vent cover”) can be configured to cover the aperture. In some examples, the first sealing member can be configured to be fixed (or not detachably coupled to) the cap 390. In some examples, the first sealing member 398 can be vapor-permeable to allow for the ingress of sterilization fluids. In some examples, the first sealing member 398 can be formed from Tyvek or any other vapor-permeable material.

The cap 390 can optionally comprise the second sealing member 399 (which is also referred to herein as a “removable sealing member,” a “second vent cover,” an “adhesive vent cover,” and/or a “removable vent cover”). The second sealing member 399 can be configured to hermetically seal the aperture in the cap 390 by covering the aperture and/or the first sealing member 398. The second sealing member 399 can comprise an adhesive cover that can be adhered to a portion of an outer or proximal surface of the cap 390 adjacent the aperture and/or the first sealing member 398, thereby forming a hermetic seal with the adjacent portions of the cap 390. In some examples the second sealing member 399 can be adhered to the cap 390 to prevent contaminants from entering the tube structure 340 after the contents of the packaging assembly 330 have been sterilized. In some examples, the second sealing member 399 can be peeled off the cap 390 to expose the aperture and/or the first sealing member 398, for example, to allow for the ingress of sterilization fluids and/or fluids for rehydrating the prosthetic heart valve 250.

Now referring to FIG. 3, some examples of the packaging assembly 330 can comprise a vapor-permeable sealing member 391 disposed between the cap 390 and the tube structure 340.

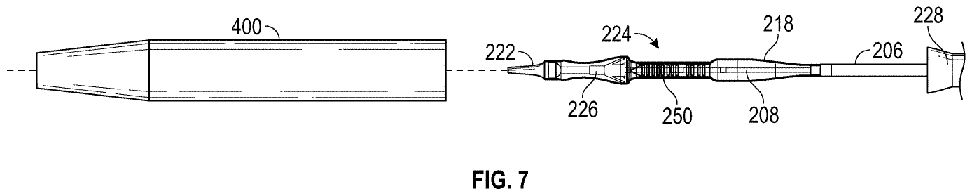

Now referring to FIG. 7, a balloon cover 400 (which is also referred to herein as a “balloon sheath” and/or a “balloon sleeve”) can comprise a sheath, sleeve, shell, or other structure configured to be inserted over and/or cover the distal end portion of the delivery apparatus 200 and/or the prosthetic heart valve 250 disposed around the distal end portion of the delivery apparatus 200. The balloon cover 400 can beneficially protect the prosthetic heart valve 250 and/or the distal portion of the delivery apparatus 200 from scratches, tears, etc., during assembly and shipping of the delivery apparatus 200. Further examples of balloon covers can be found in U.S. Pat. No. 10,588,744, which is incorporated by reference herein in its entirety.

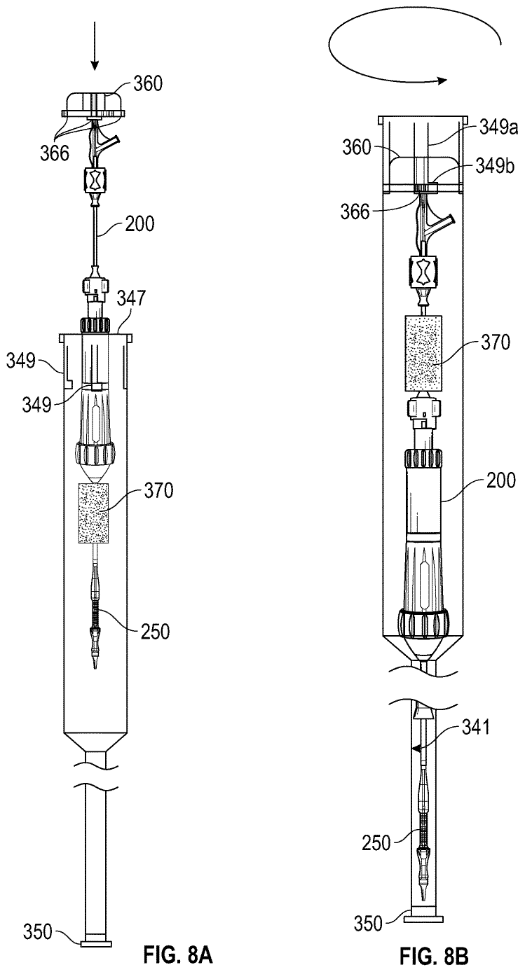

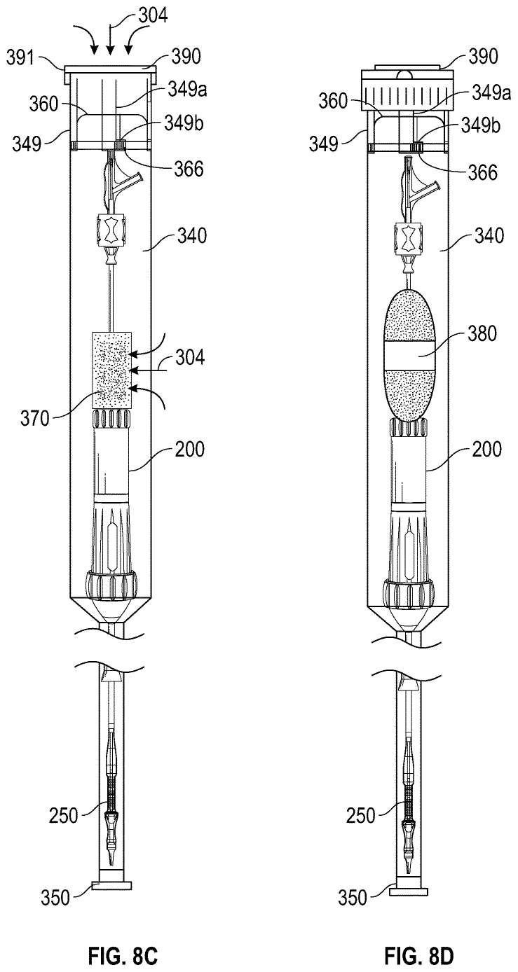

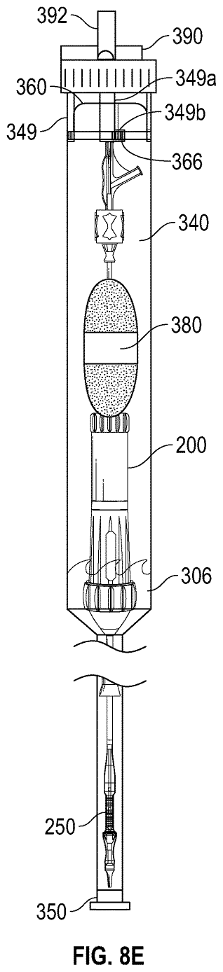

FIGS. 8A-8E are exemplary side views of the medical assembly 300. These figures illustrate a packaging process in which the prosthetic heart valve 250 and the delivery apparatus 200 are secured within the packaging assembly 330, according to one example.

FIG. 8A is a side view of the first step of the exemplary packaging process in which the delivery apparatus 200, the prosthetic heart valve 250 crimped onto the distal end portion of the delivery apparatus 200, and the retainer 360 detachably engaged with the proximal end portion of the delivery apparatus 200 are inserted into the tube structure 340 via the proximal aperture 347. The prosthetic heart valve 250 and the delivery apparatus 200 can be inserted through the proximal aperture 347 of the tube structure 340. As noted above, the retainer 360 can be coupled to the proximal end portion of the delivery apparatus by aligning the first port 238 of the delivery apparatus 200 (FIG. 3) with the second connector 368 of the retainer 360 (FIG. 5B) and then screwing the retainer 360 onto the first port 238.

FIG. 8B is a side view of the retainer 360 engaging the tube structure 340 of the packaging assembly 330, according to one example. As the delivery apparatus 200 and the retainer 360 coupled thereto are inserted into the tube structure 340, the second locking features 366 of the retainer 360 (FIG. 5B) are aligned with and inserted into respective longitudinal slots of longitudinal portions 349a of the first locking features 349 of the tube structure 340 until the second locking features 366 contact the lower circumferential ribs 353a (FIG. 4). At this point, the retainer 360 can be actuated (for example, twisted or rotated) to move the second locking features 366 of the retainer 360 into the circumferential slots of the circumferential portions 349b of the first locking features 349. This moves the retainer from an unlocked position to a locked position. In the locked position, each second locking features 366 is captured between a pair of lower and upper circumferential ribs 353a, 353b of the first locking feature 349 to prevent longitudinal movement of the retainer and the delivery apparatus within the tube structure. Further, in some examples, each second locking feature 366 can frictionally engage a pair of lower and upper circumferential ribs 353a, 353b of the first locking feature 349 to restrict inadvertent rotational movement of the retainer and the delivery apparatus within the tube structure. As further shown, the prosthetic heart valve 250 and the distal end portion of the delivery apparatus 200 are spaced apart in the radial direction from the inner surface 341 of the tube structure 340 and spaced apart in the longitudinal direction from the plug 350.

FIG. 8C is a side view of the medical assembly 300 being sterilized, according to one example. In some examples, the vapor-permeable sealing member 391 can be coupled to the tube structure 340 to cover the proximal aperture 347. Subsequently, the medical assembly 300 can be exposed to a sterilization fluid (for example, a gas comprising ethylene oxide), which can permeate through sealing members 370 and 391 in the direction of arrows 304 into the hollow cavity of the tube structure 340, thereby sterilizing the prosthetic heart valve 250 and the delivery apparatus 200.

FIG. 8D is a side view of the medical assembly 300 in a fully sealed configuration, according to one example. As shown, the second sealing member 380 can be adhered to the tube structure 340 to cover the sealing member 370 (which extends over or is located in the intermediate aperture), and the cap 390 can be coupled to the tube structure 340 to cover the aperture 347 and the sealing member 391. The preceding steps illustrated in FIGS. 8A-8C (including a step of fully or partially crimping the prosthetic valve 250 on the delivery apparatus 200) can be performed by the manufacturer. The sterilized medical assembly 300 optionally can be inserted into a flexible sterile pouch or bag prior to shipment.

FIG. 8E is a side view of the medical assembly 300 with a hydrating solution 306 disposed therein, according to one example. In some examples, immediately prior to an implantation procedure, the user (for example, a physician) can remove the cap 390 from the tube structure 340 and introduce the hydrating solution 306 (for example, a saline solution) into the tube structure to rehydrate the leaflets of the prosthetic heart valve 250 stored within the tube structure 340. If a balloon cover 400 is present, the balloon cover 400 can be removed (and can be discarded) from the delivery apparatus 200 and the prosthetic heart valve 250. A sufficient amount of the hydrating solution 306 can be poured into the tube structure 340 to fully immerse the prosthetic heart valve 250. Thereafter, the cap 390 can be placed back on the tube structure 340 while the prosthetic valve 250 is allowed to rehydrate. Furthermore, the hinged handle 392 can be extended to allow for the medical assembly 300 to be hung, for example, from a rack in a clinical environment while the prosthetic heart valve 250 is allowed to rehydrate.

Once the prosthetic heart valve 250 is rehydrated, the delivery apparatus 200 and the prosthetic valve 250 are removed from the tube structure 340 by first removing the cap 390, rotating the retainer 360 to the unlocked position, and then pulling the retainer 360 along with the delivery apparatus 200 and the prosthetic valve 250 from the tube structure 340. Thereafter, the retainer 360 can be removed from the delivery apparatus 200.

Although the medical assembly 300 is described as including the prosthetic heart valve 250 pre-crimped on the delivery apparatus 200, it should be noted that in some examples, the prosthetic heart valve 250 (or another implantable medical device to be used with the delivery apparatus 200) can be packaged separately from the delivery apparatus 200, such as in a separate storage jar.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An assembly comprising:
   a delivery apparatus comprising a proximal end portion and a distal end portion, wherein the proximal end portion comprises a first connector;
   a prosthetic heart valve crimped around the distal end portion of the delivery apparatus; and
   a packaging comprising:
   a hollow body comprising a proximal end portion and a distal end portion, wherein the delivery apparatus and prosthetic heart valve crimped thereunto are disposed within the hollow body; and
   a retainer comprising a second connector configured to detachably engage the first connector of the delivery apparatus.
Example 2. The assembly of any example herein, particularly example 1, wherein the distal end portion of the delivery apparatus can be suspended within the hollow body when the retainer is engaged with both the delivery apparatus and the hollow body and when the packaging and the delivery apparatus are placed in a vertical orientation with the proximal end portion of the hollow body above the distal end portion of the hollow body.
Example 3. The assembly of any example herein, particularly any one of examples 1-2, wherein the distal end portion of the delivery apparatus and the prosthetic heart valve can be spaced apart in a radial direction from an inner surface of the hollow body.
Example 4. The assembly of any example herein, particularly any one of examples 1-3, wherein the distal end portion of the delivery apparatus and the prosthetic heart valve can be spaced apart in a longitudinal direction from a distal end of the hollow body.
Example 5. The assembly of any example herein, particularly any one of examples 1-4, wherein the delivery apparatus can comprise a balloon, and wherein the prosthetic heart valve can be crimped around the balloon of the delivery apparatus.
Example 6. The assembly of any example herein, particularly example 5, which can further comprise a balloon cover circumferentially disposed around the balloon of the delivery apparatus and the prosthetic heart valve crimped around the balloon.
Example 7. The assembly of any example herein, particularly any one of examples 1-6, wherein the first connector of the delivery apparatus can be a port of a threaded Y-connector.
Example 8. The assembly of any example herein, particularly example 7, wherein the port can comprise a lumen for receiving a guidewire.
Example 9. The assembly of any example herein, particularly any one of examples 7-8, wherein the port can comprise external threads for engaging internal threads of the retainer.
Example 10. The assembly of any example herein, particularly any one of examples 1-9, wherein the packaging can further comprise a plug coupled to the distal end portion of the hollow body.
Example 11. The assembly of any example herein, particularly example 10, wherein the distal end portion of the delivery apparatus and the prosthetic heart valve crimped thereunto can be spaced apart in a longitudinal direction from the plug.
Example 12. The assembly of any example herein, particularly any one of examples 1-11, wherein the hollow body of the packaging can be a rigid body.
Example 13. The assembly of any example herein, particularly any one of examples 12, wherein the packaging can further comprise a cap detachably engaged with the proximal end portion of the hollow body.
Example 14. The assembly of any example herein, particularly example 13, wherein the cap can comprise a sealable vent.
Example 15. The assembly of any example herein, particularly example 14, wherein the packaging can further comprise an adhesive vent cover configured to cover the sealable vent.
Example 16. The assembly of any example herein, particularly any one of examples 1-15, wherein the assembly can be sterilized using an ethylene oxide gas.
Example 17. A packaging assembly for a delivery apparatus for an implantable medical device comprising:
   a tube structure comprising a proximal end portion defining a proximal end, a distal end portion defining a distal end, an inner surface, an outer surface, and at least one aperture; and
   a first sealing member covering the aperture, wherein the first sealing member is vapor-permeable.
Example 18. The delivery apparatus package of any example herein, particularly example 17, wherein the first sealing member can be formed from Tyvek.
Example 19. The delivery apparatus package of any example herein, particularly any one of examples 17-18, wherein the first sealing member can be covered by a removable second sealing member, and wherein the second sealing member can be vapor-impermeable.
Example 20. The delivery apparatus package of any example herein, particularly example 19, wherein the second sealing member can be peelable off the packaging assembly to expose the first sealing member.
Example 21. An assembly comprising:
   a delivery apparatus;
   an implantable medical device coupled to the delivery apparatus; and
   a packaging assembly for containing the delivery apparatus and implantable medical device therein, the packaging assembly comprising:
      a tube structure; and
      a retainer detachably coupled to an end portion of the tube structure, wherein:
         the retainer is configured to engage a proximal end portion of the delivery apparatus, and
         a distal end portion of the delivery apparatus does not directly contact an inner surface of the tube structure when the retainer engages the proximal end portion of the delivery apparatus, the delivery apparatus is disposed entirely within the tube structure, and the assembly is placed in a vertical orientation.
Example 22. The packaging assembly of any example herein, particularly example 21, wherein the tube structure can comprise a proximal end portion and a distal end portion, and wherein the retainer can be detachably coupled to the proximal end portion of the tube structure.
Example 23. The packaging assembly of any example herein, particularly example 22, which can further comprise a plug coupled to the distal end portion of the tube structure, wherein the tube structure and the plug can form a first hermetic seal.
Example 24. The packaging assembly of any example herein, particularly any one of examples 22-23, which can further comprise a cap coupled to the proximal end portion of the tube structure and covering the retainer, wherein the tube structure and the cap can form a second hermetic seal.
Example 25. The packaging assembly of any example herein, particularly example 24, which can further comprise a vapor-permeable sealing member disposed between the retainer and the cap.
Example 26. The packaging assembly of any example herein, particularly any one of examples 24-25, wherein the cap can comprise a hinged handle.
Example 27. The packaging assembly of any example herein, particularly any one of examples 24-26, wherein the cap can comprise a recess.
Example 28. The packaging assembly of any example herein, particularly example 27, wherein the cap can comprise a temperature sensor disposed in the recess of the cap.
Example 29. The packaging assembly of any example herein, particularly any one of examples 27-28, wherein the cap can comprise a humidity sensor disposed in the recess of the cap.
Example 30. A packaging assembly for a delivery apparatus for an implantable medical device comprising:
   a tube structure comprising a proximal end portion defining a proximal end, a distal end portion defining a distal end, an inner surface, an outer surface, and at least one first locking feature disposed on the inner surface along the proximal end portion;
   a retainer comprising:
      at least one second locking feature, wherein the second locking feature is configured to engage the first locking feature; and
      a connector configured to be releasably connected to a proximal end portion of the delivery apparatus.
Example 31. The packaging assembly of any example herein, particularly example 30, wherein the tube structure can comprise a proximal tube segment having a first diameter and a distal tube segment having a second diameter, wherein the first diameter can be greater than the second diameter.
Example 32. The packaging assembly of any example herein, particularly example 31, wherein the proximal tube segment and the distal tube segment can be joined to form a first hermetic seal.
Example 33. The packaging assembly of any example herein, particularly any one of examples 30-32, wherein the tube structure can comprise an aperture disposed between the proximal end thereof and the distal end thereof and extending from the inner surface to the outer surface, the aperture can be covered by a first sealing member, and the first sealing member can be vapor-permeable.
Example 34. The packaging assembly of any example herein, particularly example 33, wherein the first sealing member can be formed from Tyvek.
Example 35. The packaging assembly of any example herein, particularly any one of examples 33-34, wherein the first sealing member can be covered by a removable second sealing member, and wherein the second sealing member can be vapor-impermeable.
Example 36. The packaging assembly of any example herein, particularly example 35, wherein the second sealing member can be peelable off the packaging assembly to expose the first sealing member.
Example 37. The packaging assembly of any example herein, particularly any one of examples 30-36, which can further comprise a cap covering the proximal end of the tube structure.
Example 38. The packaging assembly of any example herein, particularly example 37, wherein the cap can engage threads disposed on the outer surface of the tube structure.
Example 39. The packaging assembly of any example herein, particularly any one of examples 30-38, wherein the first locking feature can be disposed at a location spaced apart from the proximal end of the tube structure.
Example 40. The packaging assembly of any example herein, particularly any one of examples 30-39, wherein the tube structure can comprise a plurality of first locking features and the retainer can comprise a plurality of corresponding second locking features.
Example 41. The packaging assembly of any example herein, particularly example 40, wherein the plurality of second locking features can be spaced apart from each other in a circumferential direction.
Example 42. The packaging assembly of any example herein, particularly any one of examples 30-41, wherein the first locking feature can be a locking slot and the second locking feature can be a locking lug.
Example 43. The packaging assembly of any example herein, particularly any one of examples 30-42, wherein the tube structure is straight.
Example 44. The packaging assembly of any example herein, particularly any one of examples 30-43, wherein the tube structure defines an internal cavity sized to contain the delivery apparatus in a straight configuration without any bends.
Example 45. The assembly of any example herein, wherein the assembly can be sterilized.

The features described herein with regard to any example can be combined with other features described in any one or more of the other examples, unless otherwise stated. For example, any one or more of the features of one assembly can be combined with any one or more features of another assembly. As another example, any one or more features of one package or packaging can be combined with any one or more features of another package or packaging.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. An assembly comprising:
a delivery apparatus comprising a proximal end portion and a distal end portion, wherein the proximal end portion comprises a first connector;
a prosthetic heart valve crimped around the distal end portion of the delivery apparatus; and
a packaging comprising:
a hollow body comprising a proximal end portion and a distal end portion, wherein the delivery apparatus and prosthetic heart valve crimped thereunto are disposed within the hollow body; and
a retainer comprising a second connector configured to detachably engage the first connector of the delivery apparatus.

2. The assembly of claim 1, wherein the distal end portion of the delivery apparatus is suspended within the hollow body when the retainer is engaged with both the delivery apparatus and the hollow body and when the packaging and the delivery apparatus are placed in a vertical orientation with the proximal end portion of the hollow body above the distal end portion of the hollow body.

3. The assembly of claim 1, wherein the distal end portion of the delivery apparatus and the prosthetic heart valve are spaced apart in a radial direction from an inner surface of the hollow body.

4. The assembly of claim 1, wherein the distal end portion of the delivery apparatus and the prosthetic heart valve are spaced apart in a longitudinal direction from a distal end of the hollow body.

5. The assembly of claim 1, wherein the first connector of the delivery apparatus is a port of a threaded Y-connector.

6. The assembly of claim 1, wherein the packaging further comprises a cap detachably engaged with the proximal end portion of the hollow body.

7. The assembly of claim 6, wherein the cap comprises a sealable vent.

8. The assembly of claim 7, wherein the packaging further comprises an adhesive vent cover configured to cover the sealable vent.

9. An assembly comprising:
a delivery apparatus;
an implantable medical device coupled to the delivery apparatus; and
a packaging assembly for containing the delivery apparatus and implantable medical device therein, the packaging assembly comprising:
a tube structure; and
a retainer detachably coupled to an end portion of the tube structure, wherein:
the retainer is configured to engage a proximal end portion of the delivery apparatus, and
a distal end portion of the delivery apparatus does not directly contact an inner surface of the tube structure when the retainer engages the proximal end portion of the delivery apparatus, the delivery apparatus is disposed entirely within the tube structure, and the assembly is placed in a vertical orientation.

10. The assembly of claim 9, wherein the tube structure comprises a proximal end portion and a distal end portion, and wherein the retainer is detachably coupled to the proximal end portion of the tube structure.

11. The assembly of claim 10, further comprising a cap coupled to the proximal end portion of the tube structure and covering the retainer, wherein the tube structure and the cap form a second hermetic seal.

12. The assembly of claim 11, further comprising a vapor-permeable sealing member disposed between the retainer and the cap.

13. The assembly of claim 11, wherein the cap comprises a recess.

14. The assembly of claim 13, wherein the cap comprises a temperature sensor disposed in the recess of the cap.

15. The assembly of claim 13, wherein the cap comprises a humidity sensor disposed in the recess of the cap.

16. A packaging assembly for a delivery apparatus for an implantable medical device comprising:
a tube structure comprising a proximal end portion defining a proximal end, a distal end portion defining a distal end, an inner surface, an outer surface, and at least one first locking feature disposed on the inner surface along the proximal end portion;
a retainer comprising:
at least one second locking feature, wherein the second locking feature is configured to engage the first locking feature; and
a connector configured to be releasably connected to a proximal end portion of the delivery apparatus.

17. The packaging assembly of claim 16, wherein the tube structure comprises an aperture disposed between the proximal end thereof and the distal end thereof and extending from the inner surface to the outer surface, the aperture is covered by a first sealing member, and the first sealing member is vapor-permeable.

18. The packaging assembly of claim 17, wherein the first sealing member is covered by a removable second sealing member, and wherein the second sealing member is vapor-impermeable.

19. The packaging assembly of claim 18, wherein the second sealing member is peelable off the packaging assembly to expose the first sealing member.

20. The packaging assembly of claim 16, wherein the first locking feature is disposed at a location spaced apart from the proximal end of the tube structure.

21. The packaging assembly of claim 16, wherein the tube structure comprises a plurality of first locking features and the retainer comprises a plurality of corresponding second locking features.

22. The packaging assembly of claim 21, wherein the plurality of second locking features are spaced apart from each other in a circumferential direction.
